(19) United States
(12) Patent Application Publication
(10) Pub. No.: US 2025/0114008 A1
(43) Pub. Date: Apr. 10, 2025

(54) SYSTEM AND METHOD FOR A ONE CLICK SCAN WITH CAMERA AND CALIBRATION INTEGRATED WORKFLOW

(71) Applicant: GE Precision Healthcare LLC, Waukesha, WI (US)

(72) Inventors: Dattesh Dayanand Shanbhag, Bangalore (IN); Kavitha Manickam, Pewaukee, WI (US); Dawei Gui, Sussex, WI (US); Maggie MeiKei Fung, Jersey City, NJ (US); Ting Ye, Beijing (CN); Chitresh Bhushan, Glenville, NY (US); Muhan Shao, Clifton Park, NY (US)

(21) Appl. No.: 18/483,757

(22) Filed: Oct. 10, 2023

(51) Int. Cl. A61B 5/055; A61B 5/00; G06T 7/00

(52) U.S. Cl. CPC A61B 5/055; A61B 5/0037; A61B 5/0077; A61B 5/7267; G06T 7/0012; G06T 2207/10088

(57) ABSTRACT

A method for performing a scan of a subject includes receiving a selected protocol for the scan and triggering, upon receiving a start signal, automatic landmarking of the subject on a table of a magnetic resonance imaging (MRI) scanner utilizing a three-dimensional (3D) camera. The method includes obtaining landmark positioning data from the 3D camera and utilizing the landmark positioning data for localization of the region of interest. The method includes, subsequent to the automatic landmarking, triggering a calibration scan of the subject with the MRI scanner and obtaining calibration data from the MRI scanner and utilizing the calibration data for refining the localization of the region of interest. The method includes generating a geometry plan for subsequent scans utilizing both the landmark positioning data and the calibration data and triggering at least one subsequent scan of the subject with the MRI scanner based on the geometry plan.

### BACKGROUND

The subject matter disclosed herein relates to medical imaging and, more particularly, to a system and method for a one click scan with camera and calibration integrated workflow for a magnetic resonance imaging (MRI) system.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Setting up off-isocenter anatomical scans (e.g., musculoskeletal (MSK) MRI scans (e.g., for a shoulder, biceps, limbs, a wrist, a knee, an arm, etc.)) are always a challenge and require skilled operators. For example, left versus right localization of extremities is always a challenging problem for MSK scans. Typically, protocols for these scans include obtaining and utilizing 3 plane localizers (e.g., images in the axial, sagittal, and coronal planes) which are not accurate most of the time resulting in needing to repeat the localizer scan to get the proper setup for a subsequent scan. Even though obtaining localizer images involve simple protocols, these off-isocenter anatomical scans turn into long scans, if the subject (e.g., patient) is not set up properly for the scan.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for performing a scan of a region of interest of a subject utilizing a magnetic resonance imaging (MRI) system is provided. The computer-implemented method includes receiving, via the processor, a selected protocol for the scan of the region of interest of the subject utilizing an MRI scanner of the MRI system. The computer-implemented method also includes triggering, via the processor, upon receiving a start signal automatic landmarking of the subject on a table of the MRI scanner of the MRI system utilizing a three-dimensional (3D) camera. The computer-implemented further includes obtaining, at the processor, landmark positioning data from the 3D camera and utilizing, via the processor, the landmark positioning data for localization of the region of interest. The computer-implemented method even further includes subsequent to the automatic landmarking, triggering, via the processor, a calibration scan of the subject with the MRI scanner. The computer-implemented method yet further includes obtaining, at the processor, calibration data from the MRI scanner and utilizing, via the processor, the calibration data for refining the localization of the region of interest. The computer-implemented method still further includes generating, via the processor, a geometry plan for subsequent scans of the region of interest of the subject with the MRI scanner utilizing both the landmark positioning data and the calibration data. The computer-implemented yet further includes triggering, via the processor, at least one subsequent scan of the subject with the MRI scanner based on the geometry plan.

In another embodiment, a system for performing a scan of a region of interest of a subject utilizing a magnetic resonance imaging (MRI) system is provided. The system includes a memory encoding processor-executable routines. The system also includes a processor configured to access the memory and to execute the processor-executable routines, wherein the processor-executable routines, when executed by the processor, cause the processor to perform actions. The actions include receiving a selected protocol for the scan of the region of interest of the subject utilizing an MRI scanner of the MRI system. The actions also include triggering, upon receiving a start signal, automatic landmarking of the subject on a table of the MRI scanner of the MRI system utilizing a three-dimensional (3D) camera. The actions further include obtaining landmark positioning data from the 3D camera and utilizing the landmark positioning data for localization of the region of interest. The actions even further include, subsequent to the automatic landmarking, triggering a calibration scan of the subject with the MRI scanner. The actions yet further include obtaining calibration data from the MRI scanner and utilizing the calibration data for refining the localization of the region of interest. The actions still further include generating a geometry plan for subsequent scans of the region of interest of the subject with the MRI scanner utilizing both the landmark positioning data and the calibration data. The actions yet further include triggering at least one subsequent scan of the subject with the MRI scanner based on the geometry plan.

In a further embodiment, a non-transitory computer-readable medium, the computer-readable medium including processor-executable code that when executed by a processor, causes the processor to perform actions. The actions include receiving a selected protocol for a scan of a region of interest of a subject utilizing a magnetic resonance imaging (MRI) scanner of an MRI system. The actions also include triggering, upon receiving a start signal, automatic landmarking of the subject on a table of the MRI scanner of the MRI system utilizing a three-dimensional (3D) camera. The actions further include obtaining landmark positioning data from the 3D camera and utilizing the landmark positioning data for localization of the region of interest. The actions even further include, subsequent to the automatic landmarking, triggering a calibration scan of the subject with the MRI scanner. The actions yet further include obtaining calibration data from the MRI scanner and utilizing the calibration data for refining the localization of the region of interest. The actions still further include generating a geometry plan for subsequent scans of the region of interest of the subject with the MRI scanner utilizing both the landmark positioning data and the calibration data. The actions yet further include triggering at least one subsequent scan of the subject with the MRI scanner based on the geometry plan.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique;

FIG. 2 illustrates a schematic diagram of an MRI scanner of the MRI system in FIG. 1 disposed in a scan room (e.g., having a camera coupled to the MRI scanner), in accordance with aspects of the present disclosure;

FIG. 3 illustrates a schematic diagram of an MRI scanner of the MRI system in FIG. 1 disposed in a scan room (e.g., having a camera coupled to the ceiling), in accordance with aspects of the present disclosure;

FIG. 4 illustrates a schematic diagram of a one click scan with camera and calibration integrated workflow utilizing the MRI system in FIG. 1, in accordance with aspects of the present disclosure;

FIG. 5 illustrates a flow diagram of a method for performing a scan of a region of interest of a subject (e.g., patient) utilizing the MRI system in FIG. 1 utilizing a camera and calibration integrated workflow, in accordance with aspects of the present disclosure;

FIG. 6 illustrates a flow diagram of a method for performing a scan of a region of interest of a subject (e.g., patient) utilizing the MRI system in FIG. 1 utilizing a camera and calibration integrated workflow (e.g., having a localizer and diagnostic scan), in accordance with aspects of the present disclosure;

FIG. 7 is an example of an image of a knee acquired during a calibration scan, in accordance with aspects of the present disclosure;

FIG. 8 is an example of an image of coil signal intensity information obtained during a calibration scan of the knee, in accordance with aspects of the present disclosure;

FIG. 9 is another example of an image of coil signal intensity information obtained during a calibration scan of the knee, in accordance with aspects of the present disclosure;

FIG. 10 are examples of localizer images obtained of a knee, in accordance with aspects of the present disclosure;

FIG. 11 are examples of high-resolution images obtained of a knee, in accordance with aspects of the present disclosure; and

FIG. 12 illustrates a flow diagram of a method for performing a scan of a region of interest of a subject (e.g., patient) utilizing the MRI system in FIG. 1 utilizing a camera and calibration integrated workflow (e.g., having a diagnostic scan), in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep-learning (DL) approaches discussed herein may be based on artificial neural networks, and may therefore encompass one or more of deep neural networks, fully connected networks, convolutional neural networks (CNNs), unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides systems and methods for a one click scan with camera and calibration integrated workflow for performing a scan of a region of interest of a subject (e.g., patient) utilizing a magnetic resonance imaging (MRI) system. In particular, the disclosed systems and methods include receiving, via the processor, a selected protocol for the scan of the region of interest of the subject utilizing an MRI scanner of the MRI system. The disclosed systems and methods also include triggering, via the processor, upon receiving a start signal (e.g., start input signal) automatic landmarking of the subject on a table of the MRI scanner of the MRI system utilizing a three-dimensional (3D) camera. The disclosed systems and methods further include obtaining, at the processor, landmark positioning data from the 3D camera and utilizing, via the processor, the landmark positioning data for localization of the region of interest. The disclosed systems and methods even further include subsequent to the automatic landmarking, triggering, via the processor, a calibration scan of the subject with the MRI scanner. The disclosed systems and methods yet further include obtaining, at the processor, calibration data from the MRI scanner and utilizing, via the processor, the calibration data for refining the localization of the region of interest. The disclosed systems and methods still further include generating, via the processor, a geometry plan (and prescription parameters for image scan plane) for subsequent scans of the region of interest of the subject with the MRI scanner utilizing both the landmark positioning data and the calibration data. The disclosed systems and methods yet further include triggering, via the processor, at least one subsequent scan of the subject with the MRI scanner based on the geometry plan (and prescription parameters for image scan plane).

In certain embodiments, the scan is a an off-isocenter anatomical scan of the subject. For example, the scan may be musculoskeletal MRI scans for a shoulder, biceps, limbs, a wrist, a knee, an arm, or other anatomical region. In certain embodiments, the at least one subsequent scan is a localizer scan to obtain localizer images (e.g., for the selected scan protocol). In certain embodiments, the at least one subsequent san is a diagnostic scan to obtain diagnostic images (e.g. utilizing the selected scan protocol).

In certain embodiments, the landmark positioning data includes one or more of subject position, subject orientation, body contour of the subject, surface level sub-region key points of the subject, superior/inferior coverage, right/left coverage, depth, localization of coils, and localization of blanket. In certain embodiments, the calibration data includes one or more of left/right center, anterior/posterior center, offset information from coil signal intensity information, patient size, and anatomy specific fine localization. In certain embodiments, utilizing the landmark positioning data for localization of the region of interest includes utilizing the landmark positioning data for superior/inferior localization of the region of interest, and wherein utilizing the calibration data for both anterior/posterior localization and right/left localization of the region of interest. The calibration scan also enables the detection and the determination of implants within the region of interest.

In certain embodiments, the disclosed systems and methods include providing. via the processor, a subject-perceptible command (e.g., voice command via speaker) to adjust positioning prior to triggering the calibration scan when an abnormal subject condition is detected based on the landmark positioning data. In certain embodiments, the disclosed systems and methods include providing, via the processor, a user-perceptible warning (e.g., on a display on an operator console of the MRI system) that an abnormal subject condition is detected based on the landmark positioning data precluding utilization of an intelligent prescription module (e.g., AIRₓ™ from General Electric Healthcare) which utilizes deep learning algorithms to automatically identify anatomical structures and to prescribe slices for a diagnostic scan.

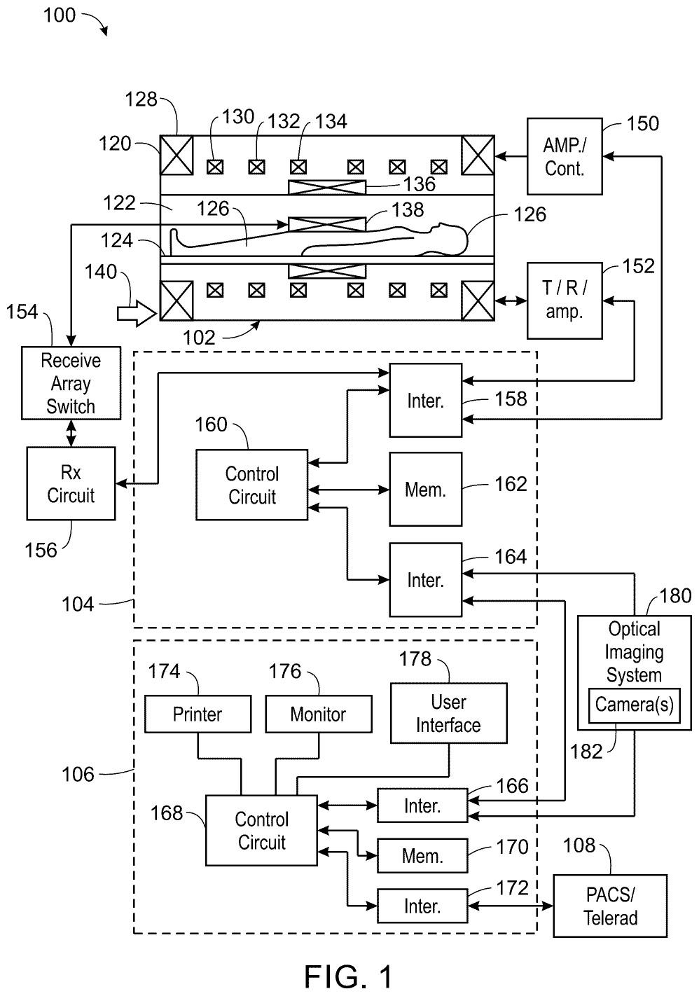

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging. It should be noted that the techniques described herein may also be utilized with imaging systems with dual imaging modalities (e.g., computed tomography (CT)/MRI system or positron emission tomography (PET)/MRI system).

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, Bo. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more neural networks for reconstruction of acquired data as described below. In certain embodiments, image reconstruction may occur on a separate computing device having processing circuitry and memory circuitry.

The programming code may enable a one click scan with camera and calibration integrated workflow for performing a scan of a region of interest of a subject 126 (e.g., patient) utilizing the MRI system 100. In particular, the programming code may enable receiving a selected protocol for the scan of the region of interest of the subject 126 utilizing an MRI scanner 102 of the MRI system 100. The programming code may enable triggering, upon receiving a start signal (e.g., start input signal), automatic landmarking of the subject on a table of the MRI scanner 102 of the MRI system 100 utilizing a three-dimensional (3D) camera. The programming code may enable obtaining landmark positioning data from the 3D camera and utilizing the landmark positioning data for localization of the region of interest. The programming code may enable, subsequent to the automatic landmarking, triggering a calibration scan of the subject 126 with the MRI scanner 102. The programming code may enable obtaining calibration data from the MRI scanner 102 and utilizing the calibration data for refining the localization of the region of interest. The programming code may enable generating a geometry plan (and prescription parameters for image scan plane) for subsequent scans of the region of interest of the subject with the MRI scanner utilizing both the landmark positioning data and the calibration data. The programming code may enable triggering at least one subsequent scan of the subject with the MRI scanner based on the geometry plan (and prescription parameters for image scan plane).

In certain embodiments, the scan is a an off-isocenter anatomical scan of the subject. For example, the scan may be musculoskeletal MRI scans for a shoulder, biceps, limbs, a wrist, a knee, an arm, or other anatomical region. In certain embodiments, the at least one subsequent scan is a localizer scan to obtain localizer images (e.g., for the selected scan protocol). In certain embodiments, the at least one subsequent san is a diagnostic scan to obtain diagnostic images (e.g. utilizing the selected scan protocol).

In certain embodiments, the landmark positioning data includes one or more of subject position, subject orientation, body contour of the subject, surface level sub-region key points of the subject, superior/inferior coverage, right/left coverage, depth, localization of coils, and localization of blanket. In certain embodiments, the calibration data includes one or more of left/right center, anterior/posterior center, offset information from coil signal intensity information, patient size, and anatomy specific fine localization. In certain embodiments, utilizing the landmark positioning data for localization of the region of interest includes utilizing the landmark positioning data for superior/inferior localization of the region of interest, and wherein utilizing the calibration data for both anterior/posterior localization and right/left localization of the region of interest. The calibration scan also enables the detection and the determination of implants within the region of interest.

In certain embodiments, the programming code may enable providing a subject-perceptible command (e.g., voice command via speaker) to adjust positioning prior to triggering the calibration scan when an abnormal subject condition is detected based on the landmark positioning data. In certain embodiments, the programming code may enable providing a user-perceptible warning (e.g., on a display on an operator console of the MRI system) that an abnormal subject condition is detected based on the landmark positioning data precluding utilization of an intelligent prescription module (e.g., AIRₓ™ from General Electric Healthcare) which utilizes deep learning algorithms to automatically identify anatomical structures and to prescribe slices for a diagnostic scan.

An additional interface circuit 172 may be provided for exchanging image data. configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

The MRI imaging system 100 also includes an optical imaging system 180. The optical imaging system 180 includes one or more cameras 182 (e.g., 3D cameras) coupled to the MRI scanner 102 or separately disposed within the a scan room having the MRI scanner 102. The one or more cameras 182 are configured to capture 3D optical imaging data of the subject 126 disposed on the table 124 prior to being moved into the bore 122 of the MRI scanner 102 for any subsequent scan (e.g., calibration scan, localizer scan, diagnostic scan, etc.). The 3D optical imaging data serves as landmark positioning data in the techniques described herein. The optical imaging system 180 is communication with the control and analysis circuit 160 and/or the control and analysis circuit 168 via the respective interfaces 164, 166.

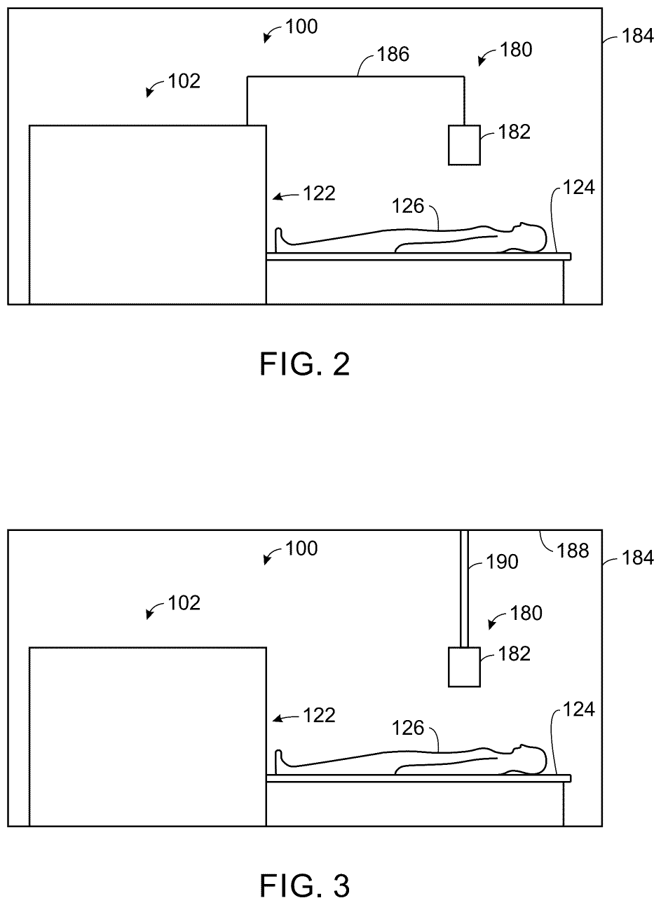

FIGS. 2 and 3 illustrates schematic diagrams of the MRI scanner 102 of the MRI system 100 in FIG. 1 disposed in a scan room 184 in conjunction with the optical imaging system 180. As depicted in FIG. 2, the camera 182 (e.g., 3D camera) of the optical imaging system 180 is coupled to the MRI scanner 102 via an extension 186 that locates the camera 182 above the subject 126 on the table 124 outside the bore 122 of the MRI scanner 102. As depicted in FIG. 3, the camera 182 is separate from the MRI scanner 102. In particular the camera 182 of the optical imaging system 180 is coupled to a ceiling 188 of the scan room 184 via an extension 190 that locates the camera 182 above the subject 126 on the table 124 outside the bore 122 of the MRI scanner 102.

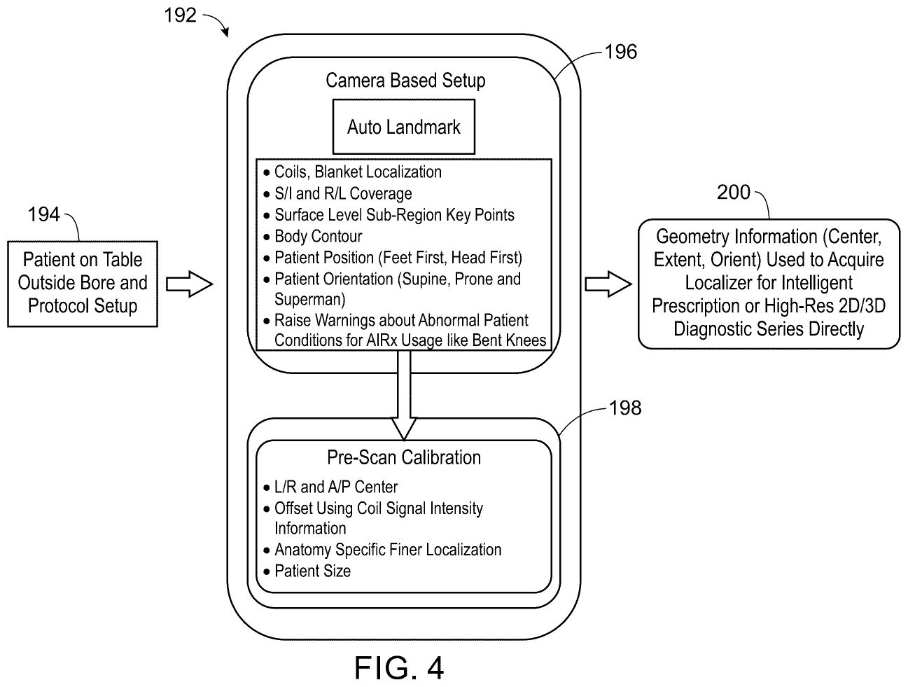

FIG. 4 illustrates a schematic diagram of a one click scan with camera and calibration integrated workflow 192 utilizing the MRI system 100 in FIG. 1. The integrated workflow 192 includes obtaining a scan protocol for imaging a region of interest of the subject (e.g., patient). The region of interest to be scanned may be obtained from electronic medical records. In certain embodiments, the scan protocol may be for an off-isocenter anatomical scan such as a musculoskeletal MRI scans of a shoulder, biceps, limbs, a wrist, a knee, an arm, or other anatomical region. The subject is placed on the table outside the bored of the MRI scanner for imaging while the scan protocol is being setup as indicated by reference numeral 194.

Upon providing an input signal (e.g., from an operator via the operator console or automatic input from door of scan room closing when operator exits the scan room), the integrated workflow 192 includes initiating and utilizing a camera based setup (e.g., optical imaging system 180 in FIG. 1) to perform automatic landmarking (e.g., localization) of the subject on the table of the MRI scanner prior to entering the bore as indicated by reference numeral 196. The 3D camera may acquire landmark positioning data for localization of the region of interest in accordance with the selected scan protocol. The landmark positioning data includes one or more of subject position, subject orientation, body contour of the subject, surface level sub-region key points of the subject, superior/inferior coverage, right/left coverage, depth, localization of coils, and localization of blanket. The camera based setup during automatic landmarking may raise warnings about abnormal patient conditions (e.g., bent knees) that may preclude utilizing an intelligent prescription module (e.g., AIRₓ™ from General Electric Healthcare). In certain embodiments, the camera based setup during automatic landmarking may provide a command to the subject (e.g., via voice command via a speaker) to adjust positioning. The camera-based system provides information about the superior/inferior location of the anatomy or region of interest (e.g., based on a model) but cannot reliably detect the right/left offset of the intended target anatomy for extremity cases. In addition, the camera-based system cannot reliably detect the anterior/posterior offset for spine/pelvis cases.

After the automatic landmarking, the integrated workflow 192 further includes (as a part of a continuous and integrated workflow in response to the start input signal) moving the patient into the bore of the MRI scanner via the table and performing a pre-scan calibration. The pre-scan calibration is utilized to obtain calibration data for refining the localization of the region of interest of the subject to be scanned. The calibration data includes one or more of left/right center, anterior/posterior center, offset information from coil signal intensity information, patient size, and anatomy specific fine localization. The calibration data can provide better right/left and anterior/posterior information from in-vivo which can be combined with the landmark positioning data. For example, the right/left information from the calibration data can be utilized for extremity cases and the anterior/posterior information can be utilized for spine/pelvis cases. In certain embodiments, utilizing the landmark positioning data for localization of the region of interest includes utilizing the landmark positioning data for superior/inferior localization of the region of interest, and wherein utilizing the calibration data for both anterior/posterior localization and/or right/left localization of the region of interest.

After the pre-scan calibration, the integrated workflow 192 even further includes generating a geometry plan (and prescription parameters for image scan plane) for subsequent scans of the region of interest of the subject with the MRI scanner utilizing both the landmark positioning data and the calibration data as indicated by reference numeral 200. The geometry plan prescribes slices for the subsequent scans. The geometry plan includes geometry information such as a center, orientation, and extent of the region of interest to be scanned. In certain embodiments, the first subsequent scan may be a localizer scan to obtain localizer images (e.g., a series of images (e.g., two-dimensional (2D) images) in the axial, sagittal, and coronal planes (e.g., 3-plane localizer scans)). In certain embodiments, the first subsequent scan may be a high-resolution diagnostic scan. A diagnostic scan is of higher resolution than a scout or localizer scan. In certain embodiments, the subsequent may include a localizer scan followed by a high-resolution scan. In certain embodiments, an intelligent prescription module (e.g., AIRₓ™ from General Electric Healthcare) may be utilized (and used the localizer images) prior to the high-resolution diagnostic scan to further refine the geometry plan and prescription of slices. In certain embodiments, during certain abnormal subject conditions (e.g., bent knees) an intelligent prescription module may not be utilized.

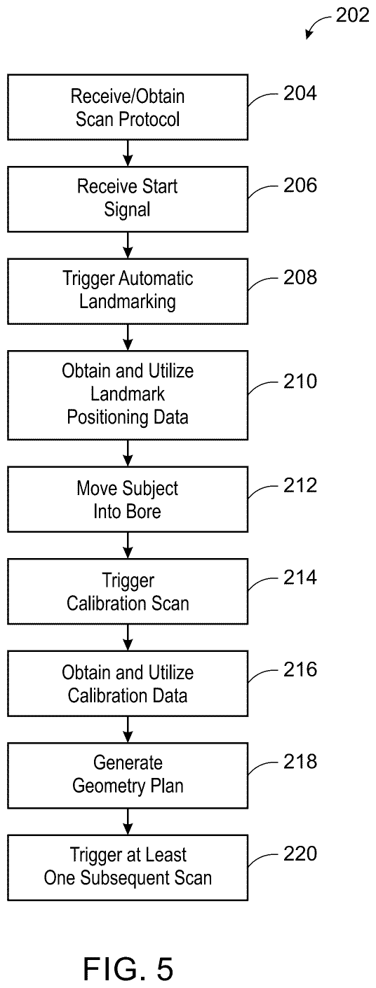

FIG. 5 illustrates a flow diagram of a method 202 for performing a scan of a region of interest of a subject (e.g., patient) utilizing the MRI system 100 in FIG. 1 utilizing a camera and calibration integrated workflow. One or more steps of the method 202 may be performed by processing circuitry of the MRI system 100 in FIG. 1. One or more of the steps of the method 202 may be performed simultaneously or in a different order from the order depicted in FIG. 5. In addition, the method 202 may also be utilized with a dual imaging modality system such as a CT/MRI system or a PET/MRI system.

Prior to the start of the method 202, a patient to be imaged is setup by a technologist on a table of the MRI scanner (e.g., MRI scanner 102 in FIG. 1). The method 202 includes receiving or obtaining a selected protocol (e.g., scan protocol) for a scan of a region of interest of a subject utilizing the MRI scanner of the MRI system (block 204). In certain embodiments, the scan and the scan protocol may be for an off-isocenter anatomical scan such as a musculoskeletal MRI scans of a shoulder, biceps, limbs, a wrist, a knee, an arm, or other anatomical region. The method 202 also includes receiving a start signal (e.g., start input signal) (block 206). The start signal may be received via an input from the technologist (e.g., via input provided via an operator console of the MRI system). The start signal may be received automatically upon closing of a door of the scan room upon the technologist leaving the scan room for the scan.

The method 202 further includes triggering (e.g., automatically triggering), upon receiving the start signal, automatic landmarking (e.g., based on the selected scan protocol) of the subject on a table of the MRI scanner of the MRI system utilizing a three-dimensional (3D) camera (e.g., of the optical imaging system 180 in FIG. 1) (block 208). The camera-based automatic landmarking occurs outside the bore of the MRI scanner. The method 202 even further includes obtaining landmark positioning data from the 3D camera and utilizing the landmark positioning data for localization of the region of interest (block 210). In certain embodiments, the landmark positioning data includes one or more of subject position, subject orientation, body contour of the subject, surface level sub-region key points of the subject, superior/inferior coverage, right/left coverage, depth, localization of coils, and localization of blanket.

The method 202 still further includes, subsequent to the automatic landmarking, moving (via the table) the subject into the bore of the MRI scanner (block 212). The method 202 yet further includes triggering (e.g., automatically triggering) a calibration scan (e.g., based on the selected scan protocol) of the subject with the MRI scanner (block 214). The method 202 further includes obtaining calibration data from the MRI scanner and utilizing the calibration data for refining the localization of the region of interest (block 216). The calibration data includes one or more of left/right center, anterior/posterior center, offset information from coil signal intensity information, patient size, and anatomy specific fine localization. The calibration data can provide better right/left and anterior/posterior information from in-vivo which can be combined with the landmark positioning data. For example, the right/left information from the calibration data can be utilized for extremity cases and the anterior/posterior information can be utilized for spine/pelvis cases. In certain embodiments, utilizing the landmark positioning data for localization of the region of interest includes utilizing the landmark positioning data for superior/inferior localization of the region of interest, and wherein utilizing the calibration data for both anterior/posterior localization and/or right/left localization of the region of interest.

The method 202 still further include generating a geometry plan (and prescription parameters for image scan plane) for subsequent scans of the region of interest of the subject with the MRI scanner utilizing both the landmark positioning data and the calibration data (block 218). The geometry plan prescribes slices for the subsequent scans. The geometry plan includes geometry information such as a center, orientation, and extent of the region of interest to be scanned. The method 202 yet further include triggering (e.g., automatically triggering) at least one subsequent scan (e.g., based on the selected scan protocol) of the subject with the MRI scanner based on the geometry plan (and prescription parameters for image scan plane) (block 220). In certain embodiments, the at least one subsequent scan is a localizer scan to obtain localizer images (e.g., a series of images (e.g., 2D images) in the axial, sagittal, and coronal planes (e.g., 3-plane localizer scans)). In certain embodiments, the at least one subsequent scan is a high-resolution diagnostic scan. A diagnostic scan is of higher resolution than a scout or localizer scan.

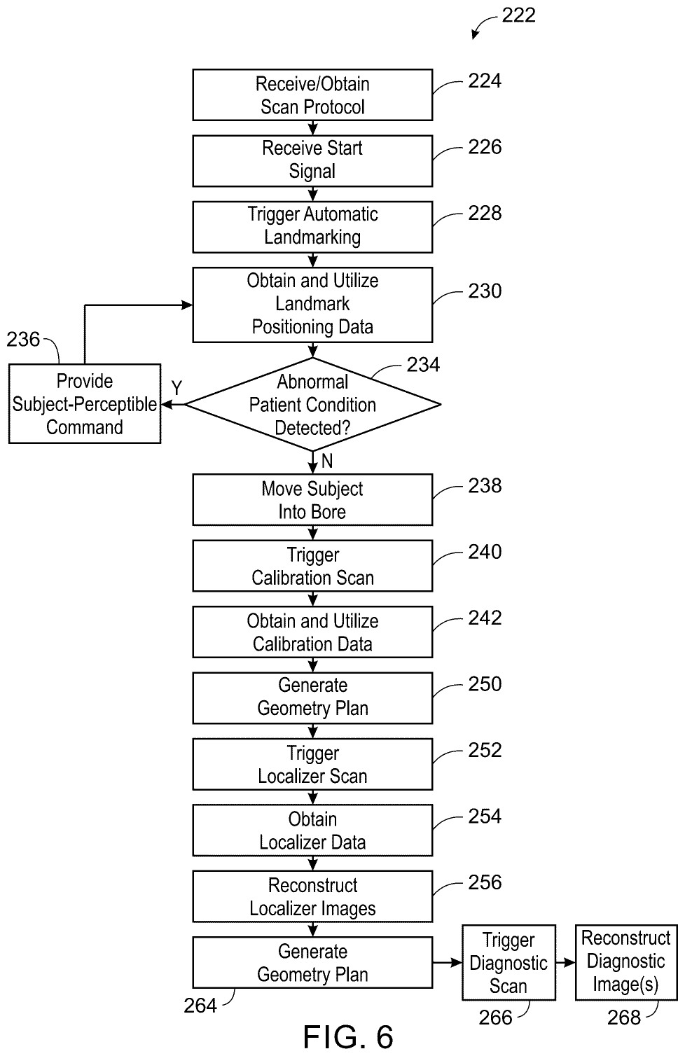

FIG. 6 illustrates a flow diagram of a method 222 for performing a scan of a region of interest of a subject (e.g., patient) utilizing the MRI system 100 in FIG. 1 utilizing a camera and calibration integrated workflow (e.g., having a localizer and diagnostic scan). One or more steps of the method 222 may be performed by processing circuitry of the MRI system 100 in FIG. 1. One or more of the steps of the method 222 may be performed simultaneously or in a different order from the order depicted in FIG. 6. In addition, the method 222 may also be utilized with a dual imaging modality system such as a CT/MRI system or a PET/MRI system.

Prior to the start of the method 222, a patient to be imaged is setup by a technologist on a table of the MRI scanner (e.g., MRI scanner 102 in FIG. 1). The method 222 includes receiving or obtaining a selected protocol (e.g., scan protocol) for a scan of a region of interest of a subject utilizing the MRI scanner of the MRI system (block 224). In certain embodiments, the scan and the scan protocol may be for an off-isocenter anatomical scan such as a musculoskeletal MRI scans of a shoulder, biceps, limbs, a wrist, a knee, an arm, or other anatomical region. The method 222 also includes receiving a start signal (e.g., start input signal) (block 226). The start signal may be received via an input from the technologist (e.g., via input provided via an operator console of the MRI system). The start signal may be received automatically upon closing of a door of the scan room upon the technologist leaving the scan room for the scan.

The method 222 further includes triggering (e.g., automatically triggering), upon receiving the start signal, automatic landmarking (e.g., based on the selected scan protocol) of the subject on a table of the MRI scanner of the MRI system utilizing a three-dimensional (3D) camera (e.g., of the optical imaging system 180 in FIG. 1) (block 228). The camera-based automatic landmarking occurs outside the bore of the MRI scanner. The method 222 even further includes obtaining landmark positioning data from the 3D camera and utilizing the landmark positioning data for localization of the region of interest (block 230). In certain embodiments, the landmark positioning data includes one or more of subject position, subject orientation, body contour of the subject, surface level sub-region key points of the subject, superior/inferior coverage, right/left coverage, depth, localization of coils, and localization of blanket.

In certain embodiments, the method 222 includes analyzing the landmark positioning data for a potential abnormal patient condition (e.g., knees bent) (block 234). If an abnormal patient condition is detected, the method 222 includes providing a subject perceptible command (e.g., voice command via a speaker) to adjust positioning prior to triggering the calibration scan (block 236). If no abnormal patient condition is detected, the method 222 includes, subsequent to the automatic landmarking, moving (via the table) the subject into the bore of the MRI scanner (block 238).

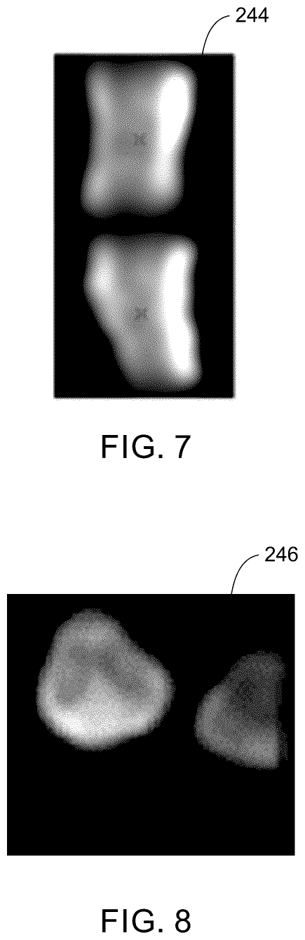

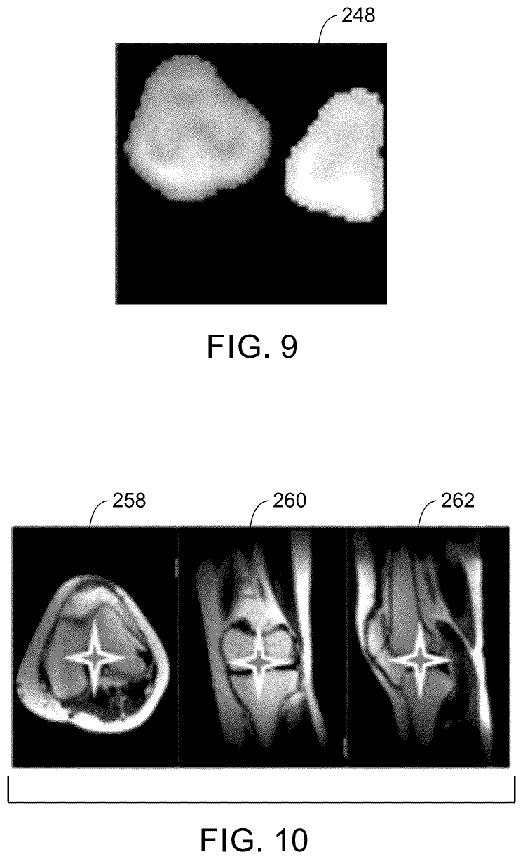

The method 222 yet further includes triggering (e.g., automatically triggering) a calibration scan (e.g., based on the selected scan protocol) of the subject with the MRI scanner (block 240). The method 222 further includes obtaining calibration data from the MRI scanner and utilizing the calibration data for refining the localization of the region of interest (block 242). FIG. 7 is an example of an image 244 of a knee acquired during a calibration scan. FIGS. 8 and 9 are examples of images 246 and 248 of coil signal intensity information obtained during a calibration scan of the knee. The coil signal intensity information may be utilized in determining or updating right/left offsets of a region of interest (e.g., the knee). Returning to FIG. 6, the calibration data includes one or more of left/right center, anterior/posterior center, offset information from coil signal intensity information, patient size, and anatomy specific fine localization. The calibration scan also enables the detection and the determination of implants within the region of interest. The calibration data can provide better right/left and anterior/posterior information from in-vivo which can be combined with the landmark positioning data. For example, the right/left information from the calibration data can be utilized for extremity cases and the anterior/posterior information can be utilized for spine/pelvis cases. In certain embodiments, utilizing the landmark positioning data for localization of the region of interest includes utilizing the landmark positioning data for superior/inferior localization of the region of interest, and wherein utilizing the calibration data for both anterior/posterior localization and/or right/left localization of the region of interest.

The method 222 still further include generating a geometry plan (and prescription parameters for image scan plane) for subsequent scans of the region of interest of the subject with the MRI scanner utilizing both the landmark positioning data and the calibration data (block 250). The geometry plan prescribes slices for the subsequent scans. The geometry plan includes geometry information such as a center, orientation, and extent of the region of interest to be scanned. The method 222 yet further include triggering (e.g., automatically triggering) a localizer scan (e.g., 3-plane localizer scan) with the MRI scanner based on the geometry plan (and prescription parameters for image scan plane) (block 252). The method 222 also includes obtaining the localizer data from the localizer scan (block 254). The method 222 further includes reconstructing localizer images from the localizer data (block 256). The localizer images are a series of images in the axial, sagittal, and coronal planes. For example, FIG. 10 depicts examples of localizer images 258, 260, and 262 of a knee obtained during a localizer scan. In particular, localizer image 258 is an axial image of the knee. Localizer image 260 is a coronal image of the knee. Localizer image 262 is a sagittal image of the knee. As depicted, the localizer images 258, 260, and 262 are correctly centered. For example, an artificial intelligence model (e.g., trained deep learning-based model) may be utilized to compute the center of the region of interest (e.g., knee) based on the landmark positioning data and the calibration data in generating the geometry plan utilized for the localizer scan.

Returning to FIG. 6, in certain embodiments, the method 222 includes generating an updated geometry plan (and prescription parameters for image scan plane) for a diagnostic scan based on the localizer images (block 264). In certain embodiments, deep learning algorithms may be utilized to automatically identify anatomical structures in the localizer images and to prescribe slices for the diagnostic scan. For example, intelligent prescription (e.g., AIRₓ™ from General Electric Healthcare) may be utilized in generating the geometry plan. In certain embodiments, statistical learning may be utilized to automatically identify anatomical structures in the localizer images and to prescribe slices for the diagnostic scan.

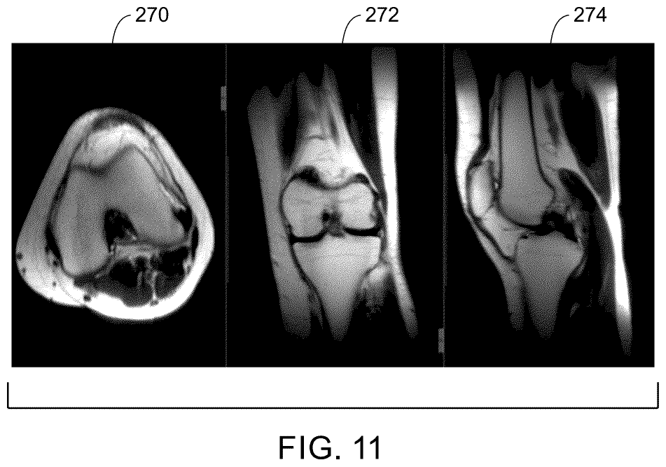

The method 222 includes triggering (e.g., automatically triggering) a diagnostic scan based on the updated geometry plan (and prescription parameters for image scan plane) (block 266). A diagnostic scan is of higher resolution than a scout or localizer scan. The method 222 also includes reconstructing one or more high-resolution diagnostic images based on image data from the diagnostic scan (block 268). FIG. 11 depicts examples of high-resolution diagnostic images 270, 272, 274 of the knee obtained during a diagnostic scan. In particular, diagnostic image 270 is an axial image of the knee. Diagnostic image 272 is a coronal image of the knee. Diagnostic image 272 is a sagittal image of the knee.

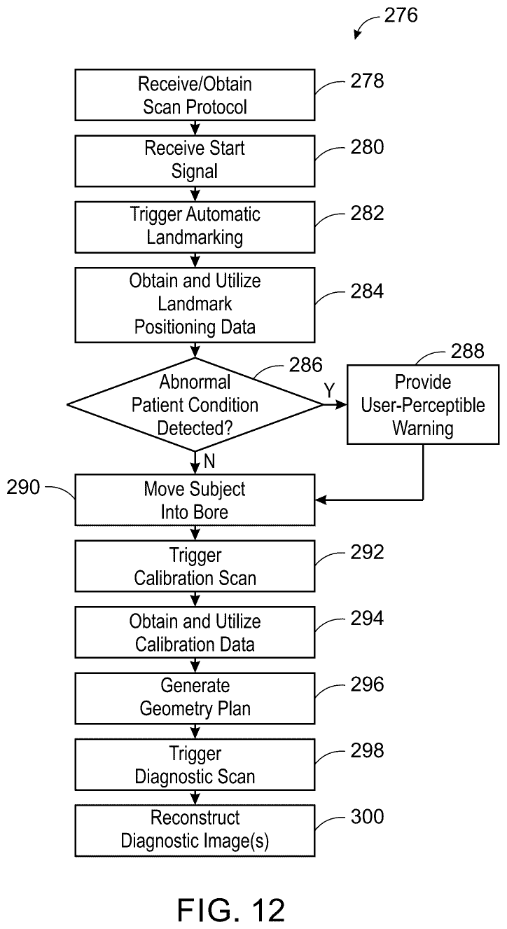

FIG. 12 illustrates a flow diagram of a method 276 for performing a scan of a region of interest of a subject (e.g., patient) utilizing the MRI system 100 in FIG. 1 utilizing a camera and calibration integrated workflow (e.g., having a diagnostic scan). One or more steps of the method 276 may be performed by processing circuitry of the MRI system 100 in FIG. 1. One or more of the steps of the method 276 may be performed simultaneously or in a different order from the order depicted in FIG. 12. In addition, the method 276 may also be utilized with a dual imaging modality system such as a CT/MRI system or a PET/MRI system.

Prior to the start of the method 276, a patient to be imaged is setup by a technologist on a table of the MRI scanner (e.g., MRI scanner 102 in FIG. 1). The method 276 includes receiving or obtaining a selected protocol (e.g., scan protocol) for a scan of a region of interest of a subject utilizing the MRI scanner of the MRI system (block 278). In certain embodiments, the scan and the scan protocol may be for an off-isocenter anatomical scan such as a musculoskeletal MRI scans of a shoulder, biceps, limbs, a wrist, a knee, an arm, or other anatomical region. The method 276 also includes receiving a start signal (e.g., start input signal) (block 280). The start signal may be received via an input from the technologist (e.g., via input provided via an operator console of the MRI system). The start signal may be received automatically upon closing of a door of the scan room upon the technologist leaving the scan room for the scan.

The method 276 further includes triggering (e.g., automatically triggering), upon receiving the start signal, automatic landmarking (e.g., based on the selected scan protocol) of the subject on a table of the MRI scanner of the MRI system utilizing a three-dimensional (3D) camera (e.g., of the optical imaging system 180 in FIG. 1) (block 282). The camera-based automatic landmarking occurs outside the bore of the MRI scanner. The method 276 even further includes obtaining landmark positioning data from the 3D camera and utilizing the landmark positioning data for localization of the region of interest (block 284). In certain embodiments, the landmark positioning data includes one or more of subject position, subject orientation, body contour of the subject, surface level sub-region key points of the subject, superior/inferior coverage, right/left coverage, depth, localization of coils, and localization of blanket.

In certain embodiments, the method 276 includes analyzing the landmark positioning data for a potential abnormal patient condition (e.g., knees bent) (block 286). If an abnormal patient condition is detected, the method 276 includes providing a user-perceptible warning (e.g., on a display on an operator console of the MRI system) that an abnormal patient condition is detected based on the landmarking positioning data precluding utilization of intelligent prescription module (e.g., AIRₓ™ from General Electric Healthcare) (block 288). If no abnormal patient condition is detected or after a warning is provided, the method 276 includes, subsequent to the automatic landmarking, moving (via the table) the subject into the bore of the MRI scanner (block 290).

The method 276 yet further includes triggering (e.g., automatically triggering) a calibration scan (e.g., based on the selected scan protocol) of the subject with the MRI scanner (block 292). The method 276 further includes obtaining calibration data from the MRI scanner and utilizing the calibration data for refining the localization of the region of interest (block 294). The calibration data includes one or more of left/right center, anterior/posterior center, offset information from coil signal intensity information, patient size, and anatomy specific fine localization. The calibration scan also enables the detection and the determination of implants within the region of interest. The calibration data can provide better right/left and anterior/posterior information from in-vivo which can be combined with the landmark positioning data. For example, the right/left information from the calibration data can be utilized for extremity cases and the anterior/posterior information can be utilized for spine/pelvis cases. In certain embodiments, utilizing the landmark positioning data for localization of the region of interest includes utilizing the landmark positioning data for superior/inferior localization of the region of interest, and wherein utilizing the calibration data for both anterior/posterior localization and/or right/left localization of the region of interest.

The method 276 still further include generating a geometry plan (and prescription parameters for image scan plane) for subsequent scans of the region of interest of the subject with the MRI scanner utilizing both the landmark positioning data and the calibration data (block 296). The geometry plan prescribes slices for the subsequent scans. The geometry plan includes geometry information such as a center, orientation, and extent of the region of interest to be scanned.

The method 276 includes triggering (e.g., automatically triggering) a diagnostic scan based on the geometry plan (and prescription parameters for image scan plane) (block 298). The method 276 proceeds to the diagnostic scan without a localizer scan. The method 276 also includes reconstructing one or more high-resolution diagnostic images based on image data from the diagnostic scan (block 300).

Technical effects of the disclosed subject matter include providing for a one click scan with camera and calibration integrated workflow for performing a scan of a region of interest of a subject (e.g., patient) utilizing an MRI system. In particular, the disclosed subject matter combines camera-based surface patient localization with calibration scan-based refinement of in vivo anatomical landmarks to obtain tailored localizers or high-resolution scans in a first go following patient setup. Specifically, camera-based automatic landmarking and smart prescription provide surface level dynamic anatomy detection, superior/inferior coverage, right/left coverage, patient position, and patient information as well as information about blankets, coils and depth information. The calibration scan provides exact localization for right/left and anterior/posterior centers and offsets, coil sensitivities, orientation of precise anatomical landmarks, and the ability to detect and determine implants. Technical effects of the disclosed subject matter also includes providing shorter scan times (especially for off-isocenter anatomical scan such as an MSK scan). Technical effects of the disclosed subject matter further include providing for a more accurate setup and prescription.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for performing a scan of a region of interest of a subject utilizing a magnetic resonance imaging (MRI) system, comprising:
receiving, via a processor, a selected protocol for the scan of the region of interest of the subject utilizing an MRI scanner of the MRI system;
triggering, via the processor, upon receiving a start signal automatic landmarking of the subject on a table of the MRI scanner of the MRI system utilizing a three-dimensional (3D) camera;
obtaining, at the processor, landmark positioning data from the 3D camera and utilizing, via the processor, the landmark positioning data for localization of the region of interest;
subsequent to the automatic landmarking, triggering, via the processor, a calibration scan of the subject with the MRI scanner;
obtaining, at the processor, calibration data from the MRI scanner and utilizing, via the processor, the calibration data for refining the localization of the region of interest;
generating, via the processor, a geometry plan for subsequent scans of the region of interest of the subject with the MRI scanner utilizing both the landmark positioning data and the calibration data; and
triggering, via the processor, at least one subsequent scan of the subject with the MRI scanner based on the geometry plan.

2. The computer-implemented method of claim 1, wherein the scan comprises an off-isocenter anatomical scan of the subject.

3. The computer-implemented method of claim 2, wherein the at least one subsequent scan comprises a localizer scan to obtain localizer images.

4. The computer-implemented method of claim 2, wherein the at least one subsequent scan comprises a diagnostic scan to obtain diagnostic images.

5. The computer-implemented method of claim 1, wherein the landmark positioning data comprises one or more of subject position, subject orientation, body contour of the subject, surface level sub-region key points of the subject, superior/inferior coverage, right/left coverage, depth, localization of coils, and localization of blanket.

6. The computer-implemented method of claim 5, wherein the calibration data comprises one or more of left/right center, anterior/posterior center, offset information from coil signal intensity information, patient size, and anatomy specific fine localization.

7. The computer-implemented method of claim 1, wherein utilizing the landmark positioning data for localization of the region of interest comprises utilizing the landmark positioning data for superior/inferior localization of the region of interest, and wherein utilizing the calibration data for both anterior/posterior localization and right/left localization of the region of interest.

8. The computer-implemented method of claim 1, further comprising providing, via the processor, a subject-perceptible command to adjust positioning prior to triggering the calibration scan when an abnormal subject condition is detected based on the landmark positioning data.

9. The computer-implemented method of claim 1, further comprising providing, via the processor, a user-perceptible warning that an abnormal subject condition is detected based on the landmark positioning data precluding utilization of an intelligent prescription module.

10. A system for performing a scan of a region of interest of a subject utilizing a magnetic resonance imaging (MRI) system, comprising:
a memory encoding processor-executable routines; and
a processor configured to access the memory and to execute the processor-executable routines, wherein the processor-executable routines, when executed by the processor, cause the processor to:
receive a selected protocol for the scan of the region of interest of the subject utilizing an MRI scanner of the MRI system;
trigger, upon receiving a start signal, automatic landmarking of the subject on a table of the MRI scanner of the MRI system utilizing a three-dimensional (3D) camera;
obtain landmark positioning data from the 3D camera and utilize the landmark positioning data for localization of the region of interest;
subsequent to the automatic landmarking, trigger a calibration scan of the subject with the MRI scanner;
obtain calibration data from the MRI scanner and utilize the calibration data for refining the localization of the region of interest;
generate a geometry plan for subsequent scans of the region of interest of the subject with the MRI scanner utilizing both the landmark positioning data and the calibration data; and
trigger at least one subsequent scan of the subject with the MRI scanner based on the geometry plan.

11. The system of claim 10, wherein the scan comprises an off-isocenter anatomical scan of the subject.

12. The system of claim 11, wherein the at least one subsequent scan comprises a localizer scan to obtain localizer images.

13. The system of claim 11, wherein the at least one subsequent scan comprises a diagnostic scan to obtain diagnostic images.

14. The system of claim 10, wherein the landmark positioning data comprises one or more of subject position, subject orientation, body contour of the subject, surface level sub-region key points of the subject, superior/inferior coverage, right/left coverage, depth, localization of coils, and localization of blanket.

15. The system of claim 14, wherein the calibration data comprises one or more of left/right center, anterior/posterior center, offset information from coil signal intensity information, patient size, and anatomy specific fine localization.

16. The system of claim 10, wherein utilizing the landmark positioning data for localization of the region of interest comprises utilizing the landmark positioning data for superior/inferior localization of the region of interest, and wherein utilizing the calibration data for both anterior/posterior localization and right/left localization of the region of interest.

17. The system of claim 10, wherein the processor-executable routines, when executed by the processor, further cause the processor to provide a subject-perceptible command to adjust positioning prior to triggering the calibration scan when an abnormal subject condition is detected based on the landmark positioning data.

18. The system of claim 10, wherein the processor-executable routines, when executed by the processor, further cause the processor to provide a user-perceptible warning that an abnormal subject condition is detected based on the landmark positioning data precluding utilization of an intelligent prescription module.

19. A non-transitory computer-readable medium, the non-transitory computer-readable medium comprising processor-executable code that when executed by a processor, causes the processor to:
receive a selected protocol for a scan of a region of interest of a subject utilizing a magnetic resonance imaging (MRI) scanner of an MRI system;
trigger, upon receiving a start signal, automatic landmarking of the subject on a table of the MRI scanner of the MRI system utilizing a three-dimensional (3D) camera;
obtain landmark positioning data from the 3D camera and utilize the landmark positioning data for localization of the region of interest;
subsequent to the automatic landmarking, trigger a calibration scan of the subject with the MRI scanner;
obtain calibration data from the MRI scanner and utilize the calibration data for refining the localization of the region of interest;
generate a geometry plan for subsequent scans of the region of interest of the subject with the MRI scanner utilizing both the landmark positioning data and the calibration data; and
trigger at least one subsequent scan of the subject with the MRI scanner based on the geometry plan.

20. The non-transitory computer-readable medium of claim 19, wherein utilizing the landmark positioning data for localization of the region of interest comprises utilizing the landmark positioning data for superior/inferior localization of the region of interest, and wherein utilizing the calibration data for both anterior/posterior localization and right/left localization of the region of interest.
